(19) United States
(12) Patent Application Publication
(10) Pub. No.: US 2019/0247415 A1
(43) Pub. Date: Aug. 15, 2019

(54) METHODS AND MATERIALS FOR ACTIVATING MUSCLE REMODELING

(71) Applicant: MAYO FOUNDATION FOR MEDICAL EDUCATION AND RESEARCH, Rochester, MN (US)

(72) Inventors: Stephen C. EKKER, Rochester, MN (US); Noriko UMEMOTO, Rochester, MN (US)

(73) Assignee: Mayo Foundation for Medical Education and Research, Rochester, MN (US)

(21) Appl. No.: 16/317,662

(22) PCT Filed: Jul. 10, 2017
(86) PCT No.: PCT/US2017/041361

§ 371 Date: Jan. 14, 2019

Related U.S. Application Data
(60) Provisional application No. 62/362,482, filed on Jul. 14, 2016.

(51) Int. Cl. A61K 31/7105; A61P 21/00; C12N 15/86

(52) U.S. Cl. CPC A61K 31/7105; A61P 21/00; C12N 2310/14; C12N 2310/20; C12N 15/86

(57) ABSTRACT

This document provides methods and materials for activating muscle remodeling. For example, methods and materials for using GAR1 inhibitors to activate muscle remodeling are provided.

### CROSS-REFERENCE TO RELATED APPLICATIONS

This application claims priority to U.S. Application Ser. No. 62/362,482, filed on Jul. 14, 2016. The disclosure of the prior application is considered part of the disclosure of this application, and is incorporated in its entirety into this application.

### STATEMENT AS TO FEDERALLY SPONSORED RESEARCH

This invention was made with government support under GM063904 awarded by the National Institutes of Health. The government has certain rights in the invention.

### BACKGROUND

### 1. Technical Field

This document relates to methods and materials involved in activating muscle remodeling. For example, this document provides methods and materials for using GAR1 ribonucleoprotein (also referred to as GAR1) inhibitors or the disruption of GAR1 expression to activate muscle remodeling.

### 2. Background Information

Muscle tissue (e.g., skeletal muscle tissue) is dynamic and responds to use. Tcap (also known as telethonin) is a polypeptide that is expressed in cardiac and skeletal muscle. It regulates sarcomere assembly, T-tubule function, and apoptosis, and mutated version of Tcap appear involved in diseases such as limb-girdle muscular dystrophy, hypertrophic cardiomyopathy, dilated cardiomyopathy, and idiopathic cardiomyopathy.

### SUMMARY

This document provides methods and materials for activating muscle remodeling. For example, this document provides methods and materials for using GAR1 inhibitors or the disruption of GAR1 expression to activate muscle remodeling. As described herein, reducing GAR1 expression or activity can increase Tcap polypeptide expression in muscle tissue and/or promote muscle remodeling. In some cases, reducing GAR1 expression or activity can increase Tcap polypeptide expression in muscle tissue and/or promote muscle remodeling with minimal or no muscle activation (e.g., excessive muscle use).

In general, one aspect of this document features a method for increasing Tcap expression within a mammal. The method comprises, or consists essentially of, (a) identifying a mammal as being in need of increased Tcap expression, and (b) administering a GAR1 inhibitor to the mammal under conditions wherein expression of Tcap within muscle cells of the mammal increases. The mammal can be a human. The GAR1 inhibitor can be an siRNA molecule.

In another aspect, this document features a method for promoting muscle remodeling in a mammal. The method comprises, or consists essentially of, (a) identifying a mammal as being in need of muscle remodeling, and (b) administering a GAR1 inhibitor to the mammal under conditions wherein muscle remodeling with the mammal is increased. The mammal can be a human. The muscle remodeling can be increased in the absence of muscle activation. The GAR1 inhibitor can be an siRNA molecule.

In another aspect, this document features a method for increasing Tcap expression within a mammal. The method comprises, or consists essentially of, (a) identifying a mammal as being in need of increased Tcap expression, and (b) disrupting endogenous nucleic acid encoding a GAR1 polypeptide within the mammal under conditions wherein expression of biologically active GAR1 polypeptides is reduced and expression of Tcap within muscle cells of the mammal increases. The mammal can be a human. The method can comprise administering a TALEN or CRISPR/Cas9 nuclease system to the mammal to disrupt the endogenous nucleic acid.

In another aspect, this document features a method for promoting muscle remodeling in a mammal, wherein the method comprises, or consists essentially of, (a) identifying a mammal as being in need of muscle remodeling, and (b) disrupting endogenous nucleic acid encoding a GAR1 polypeptide within the mammal under conditions wherein muscle remodeling with the mammal is increased. The mammal can be a human. The method can comprise administering a TALEN or CRISPR/Cas9 nuclease system to the mammal to disrupt the endogenous nucleic acid. The muscle remodeling can be increased in the absence of muscle activation.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 contains photographs of the indicated fish stained for expression of endogenous Tcap RNA using whole mount in situ hybridization.

FIG. 2 contains 4 bar graphs of relative Tcap expression compared with wild type zebrafish larvae under both normal conditions (E2 medium) and two types of forced exercise conditions (chronic physical restraint by embedding in 1% low melting agarose gel, FIGS. 2A and 2B; and intense locomotor activation induced by pentylenetetrazole (PTZ), which is a convulsant agent, FIGS. 2C and 2D).

FIG. 3 contains two bar graphs of relative Tcap expression compared with wild type zebrafish larvae under both normal conditions (E2 medium) and low dose tricaine, which is an anesthetic.

### DETAILED DESCRIPTION

This document provides methods and materials involved in promoting muscle remodeling. For example, this document provides methods and materials for using GAR1 inhibitors (e.g., siRNA molecules) or a disruption of GAR1 expression (e.g., GAR1 gene knock out) to increase Tcap polypeptide expression in muscle tissue and/or promote muscle remodeling.

Any type of animal or mammal can be treated as described herein to increase Tcap polypeptide expression in muscle tissue and/or to promote muscle remodeling. For example, humans and other primates such as monkeys can be treated with one or more GAR1 inhibitors (or treated in a manner that disrupts GAR1 expression) to increase Tcap polypeptide expression in muscle tissue and/or to promote muscle remodeling. In some cases, dogs, cats, horses, cows, pigs, sheep, mice, and rats can be treated with one or more GAR1 inhibitors (or treated in a manner that disrupts GAR1 expression) as described herein to increase Tcap polypeptide expression in muscle tissue and/or to promote muscle remodeling.

Any appropriate method can be used to identify an animal or mammal having a need for increased muscle remodeling. For example, measurements of muscle mass can be used to identify a human having a need for muscle remodeling. In some cases, the rapid loss of skeletal muscle by astronauts in zero-gravity can be reduced or minimized using the methods and materials provided herein.

Once identified as having a need for muscle remodeling, the animal or mammal can be administered or instructed to self-administer one or more GAR1 inhibitors (or an agent designed to disrupt GAR1 expression). Examples of GAR1 inhibitors include, without limitation, siRNA, shRNA, morpholino phosphorodiamidate oligonucleotides (e.g., morpholinos), small molecule inhibitors, polypeptides having GAR1 binding activity, and anti-GAR1 antibodies (or anti-GAR1 antibody like molecules). For example, siRNA or shRNA can be designed to target nucleic acid encoding a GAR1 and trigger RNA interference against GAR1 expression. Examples of agents designed to disrupt GAR1 expression include, without limitation, TALENs, TALEN variants (e.g., TALENs with cell penetrating peptides), and CRISPR/Cas9 nucleases. For example, TALENs, TALEN variants, or CRISPR/Cas9 nucleases can be designed to target nucleic acid encoding a GAR1 and introduce one or more genetic disruptions (e.g., a frame-shift or pre-mature stop codon) into the nucleic acid encoding GAR1 such that little or no GAR1 activity is observed. In some cases, a human GAR1 nucleic acid sequence can be used to design an siRNA or an shRNA that targets GAR1 nucleic acid and triggers RNA interference against GAR1 nucleic acid expression (or to design a TALEN, TALEN variant, or CRISPR/Cas9 nuclease that targets GAR1 nucleic acid and disrupts the expression of active GAR1 polypeptides). A human GAR1 nucleic acid can be as set forth in Ensembl Accession No. ENSG00000173991, GenBank Accession No. NM_018983.3 (GI No. 77812667), or GenBank Accession No. NM_032993.2 (GI No. 77812668).

Examples of siRNA, shRNA, and CRISPR/Cas9 nuclease molecules that can be used to target GAR1 nucleic acid are available commercially (see, e.g., Origene catalog numbers: TG302921, TF302921, TL302921V, SR310068, SR310068, TG514211, KN201481, and KN306306; and Biorbyt catalog number: orb270554).

Any appropriate method can be used to design an siRNA or an shRNA that targets GAR1 nucleic acid and triggers RNA interference against GAR1 nucleic acid expression. For example, software programs such as those described elsewhere (see, e.g., Naito et al., Nucleic Acids Res., 32 (Web Server issue):W124-W129 (2004)) can be used to design an siRNA or an shRNA that targets GAR1 nucleic acid (e.g., human GAR1 nucleic acid) and triggers RNA interference against GAR1 nucleic acid expression (e.g., human GAR1 nucleic acid expression). Any appropriate method can be used to design a TALEN, TALEN variant, or CRISPR/Cas9 nuclease that targets GAR1 nucleic acid and disrupts GAR1 expression.

Once designed, a particular siRNA or shRNA can be assessed in vitro or in vivo to confirm its ability to trigger RNA interference against GAR1 nucleic acid expression (e.g., human GAR1 nucleic acid expression). For example, a particular siRNA or shRNA can be administered to a mammal, and the level of GAR1 nucleic acid or GAR1 polypeptide expression within the mammal (or particular tissues or cells of the mammal) can be assessed before and after administration to identify those siRNA or shRNA molecules having the ability to trigger RNA interference against GAR1 nucleic acid expression. In some cases, once designed, a particular TALEN, TALEN variant, or CRISPR/Cas9 nuclease can be assessed in vitro or in vivo to confirm its ability to target GAR1 nucleic acid and disrupt expression of functional GAR1 polypeptides.

Any appropriate method can be used to deliver one or more siRNA or shRNA molecules (or TALEN, TALEN variant, or CRISPR/Cas9 nuclease) provided herein to cells or tissue (e.g., muscle cells or tissue) within a mammal. For example, siRNA or shRNA having the ability to trigger RNA interference against GAR1 nucleic acid expression can be configured into lipid nanoparticles such as those described elsewhere (e.g., U.S. Patent Application Publication No. 2011/0224447) to deliver the siRNA or shRNA to cells within a mammal (e.g., a human). In some cases, one or more siRNA and/or shRNA molecules having the ability to trigger RNA interference against GAR1 nucleic acid expression provided herein can be delivered to muscle cells within a mammal to treat, for example, limb-girdle muscular dystrophy, hypertrophic cardiomyopathy, dilated cardiomyopathy, idiopathic cardiomyopathy, muscular dystrophy, Pompe disease, dystrophin based dystrophies, dysferlinopathy, caveolinopathies, sarcopenia, pathogen based muscle weakening, and other diseases or conditions that lead to forms of muscle atrophy, degeneration, or weakness. By enhancing muscle remodeling, GAR1 inhibition or disruption can be therapeutically effective to address various forms of muscular degeneration.

In some cases, cyclodextrin compositions such as those described elsewhere (e.g., Arima et al., Curr. Top. Med. Chem., 14(4):465-77 (2014)) can be used to deliver one or more siRNA and/or shRNA molecules having the ability to trigger RNA interference against GAR1 nucleic acid expression to cells. In some cases, a biodegradable polymeric matrix such as those described elsewhere (e.g., Ramot et al., Toxicol Pathol., May 4 (2016) or Golan et al., Oncotarget., 6(27):24560-70 (2015)) can be used to deliver one or more siRNA and/or shRNA molecules having the ability to trigger RNA interference against GAR1 nucleic acid expression to cells.

As described herein, a composition can be formulated to contain one or more siRNA and/or shRNA molecules having the ability to trigger RNA interference against GAR1 nucleic acid expression (e.g., a composition can be formulated to contain one or more siRNA and/or shRNA molecules having the ability to trigger RNA interference against GAR1 nucleic acid expression in combination with a deliver vehicle such as a lipid nanoparticle, N-acetyl-d-galactosamine, cyclodextrin, and/or biodegradable polymeric matrix such as those described above). Such a composition containing one or more siRNA and/or shRNA molecules having the ability to trigger RNA interference against GAR1 nucleic acid expression can be administered to a mammal to increase Tcap polypeptide expression in muscle tissue and/or promote muscle remodeling. In some cases, a composition can be formulated to contain one or more TALENs, TALEN variants, or CRISPR/Cas9 nucleases designed to disrupt GAR1 expression.

In some cases, a nucleic acid molecule can be designed to express an siRNA and/or shRNA molecule having the ability to trigger RNA interference against GAR1 nucleic acid expression (or to express one or more TALENs, TALEN variants, or CRISPR/Cas9 nucleases designed to disrupt GAR1 expression). For example, a viral vector can be constructed to encode an siRNA and/or shRNA molecule having the ability to trigger RNA interference against GAR1 nucleic acid expression. In some cases, a viral vector can be constructed to encode a TALEN, TALEN variant, or CRISPR/Cas9 nuclease system designed to disrupt GAR1 expression.

In the expression vectors provided herein, a nucleic acid (e.g., a nucleic acid encoding an siRNA and/or shRNA molecule having the ability to trigger RNA interference against GAR1 nucleic acid expression, or a nucleic acid encoding a TALEN, TALEN variant, or CRISPR/Cas9 nuclease system designed to disrupt GAR1 expression) can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 to 500 nucleotides upstream of the point at which transcription starts (generally near the initiation site for RNA polymerase II).

Suitable expression vectors include, without limitation, plasmids and viral vectors derived from, for example, bacteriophage, baculoviruses, tobacco mosaic virus, herpes viruses, cytomegalovirus, retroviruses, lentiviruses, vaccinia viruses, adenoviruses, adeno-associated viruses, Semliki Forest viruses (SFV), Sindbis viruses (SIN), Venezuelan Equine Encephalitis (VEE) viruses, mango viruses, minute viruses of mice (MVM), and those described elsewhere (Stone, Viruses, 2(4):1002-1007 (2010)).

In some cases, an expression vector such as pTAT-HA, pGEX4T2, or pSF-CMV-Neo can be used to deliver an siRNA and/or shRNA molecule described herein to an animal or mammal (e.g., a human, a rodent such as a mouse or rat, a dog, a cat, a pig, a bovine species, or a horse) to be treated. Numerous vectors and expression systems are commercially available from such corporations as Novagen (Madison, Wis.), Clonetech (Palo Alto, Calif.), Stratagene (La Jolla, Calif.), and Invitrogen/Life Technologies (Carlsbad, Calif.).

In some cases, gene gun techniques, nanoparticles, electroporation methods, naked plasmid delivery methods, chitosan delivery systems, lipid systems, ultrasound based systems, PEI, lipofectamine, and other polypeptide or polymer based systems can be used to introduce nucleic acid into cells.

Any appropriate method can be used to formulate an siRNA and/or shRNA molecule having the ability to trigger RNA interference against GAR1 nucleic acid expression (or TALEN, TALEN variant, or CRISPR/Cas9 nuclease systems), or a nucleic acid encoding such an siRNA and/or shRNA molecule (or TALEN, TALEN variant, or CRISPR/Cas9 nuclease system), into a therapeutic composition. In addition, any appropriate method can be used administer such a therapeutic composition to an animal or mammal as described herein. Dosages typically are dependent on the responsiveness of the mammal to the therapeutic composition, with the course of treatment lasting from several days to several months, or until a suitable response is achieved. Optimum dosages can vary depending on the relative potency of a therapeutic composition, and generally can be estimated based on those levels found to be effective in in vitro and/or in vivo animal models.

Dosage optimization can be achieved through the application of inducible promoters to give further control of siRNA expression. Inducible vector systems that can be used as described herein include, without limitation, tetracycline inducible systems (e.g., tetOn and tetOff systems), rapamycin inducible systems, myxovirus resistance promoters, arabinose inducible promoters, iptg inducible promoters, and other systems that allow for modulating expression via temperature, light, drug delivery, or other external methods.

Therapeutic compositions provided herein may be given once or more daily, weekly, monthly, or even less often, or can be administered continuously for a period of time (e.g., hours, days, or weeks).

An siRNA and/or shRNA molecule described herein, or nucleic acid encoding an siRNA and/or shRNA molecule described herein, can be admixed, encapsulated, conjugated or otherwise associated with other molecules, molecular structures, or mixtures of compounds such as, for example, liposomes, receptor or cell targeted molecules, or oral, topical or other formulations for assisting in uptake, distribution and/or absorption.

The invention will be further described in the following examples, which do not limit the scope of the invention described in the claims.

### EXAMPLES

### Example 1—Reducing Expression of GAR1 Ribonucleoprotein Involved in a Small Nucleolar RNA (snoRNA) Increases Tcap Expression

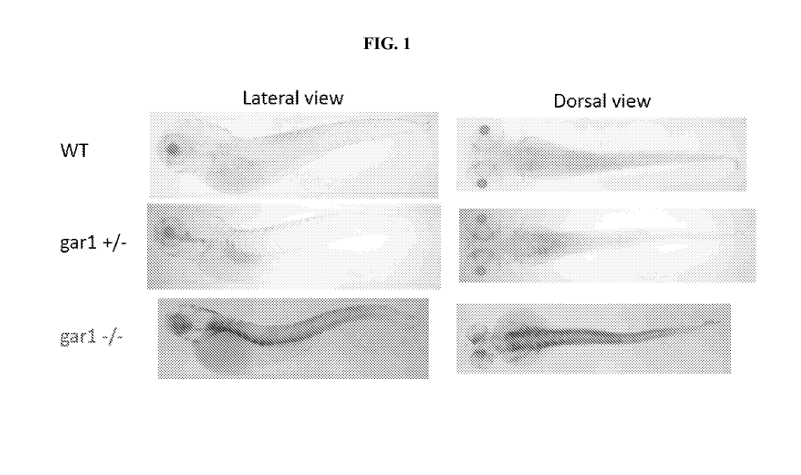

Wild type, heterozygous GAR1 knockout (gar1^{-/+}), and homozygous GAR1 knockout (gar1^{-/-}) zebrafish were obtained and assessed for tcap expression using whole mount in situ hybridization. Homozygous GAR1 knockout (gar1^{-/-}) zebrafish exhibited increased tcap expression as compared to the levels observed in wild type and heterozygous GAR1 knockout (gar1^{-/+}) zebrafish (FIG. 1). These results were obtained with little or no muscle activity indicating that reduced expression of GAR1 ribonucleoprotein, which is involved in H/ACA snoRNA, can increase tcap expression in the absence of muscle activation.

### Example 2—Muscle Activation with Forced Exercises Induced by Physical Resistant in Low Melting Agarose Gel Increases Tcap Expression

Wild type zebrafish at 2, 3, 4, and 5 dpf under normal conditions (in E2 medium) and embedded in low melting agarose gel were assessed for tcap expression using quantitative RT-PCR.

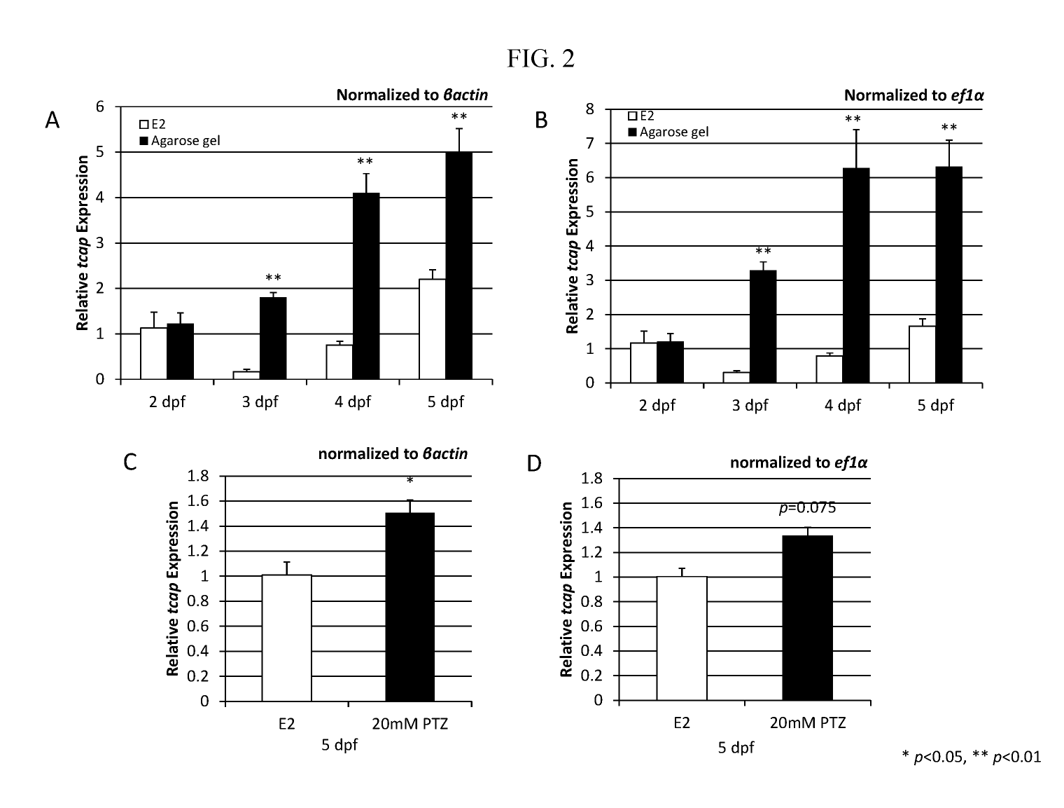

The wild type zebrafish embedded in low melting agarose exhibited significantly increased Tcap expression as compared to the levels observed in wild type zebrafish under the normal conditions (FIGS. 2A and 2B). These results were obtained with muscle activity indicating that physical resistant in agarose gel can increase tcap expression.

### Example 3—Muscle Activation with Forced Exercises Induced by PTZ Increases Tcap Expression

Wild type zebrafish at 5 dpf under normal conditions (in E2 medium) and treated with 20 mM PTZ were assessed for tcap expression using quantitative RT-PCR. The wild type zebrafish treated with 20 mM PTZ exhibited significantly increased Tcap expression (FIGS. 2C and 2D). These results were obtained with muscle activity indicating that intense locomotor activity induced by PTZ treatment can increase tcap expression.

### Example 4—Blocked Muscle Activation with Anesthesia Decreases Tcap Expression

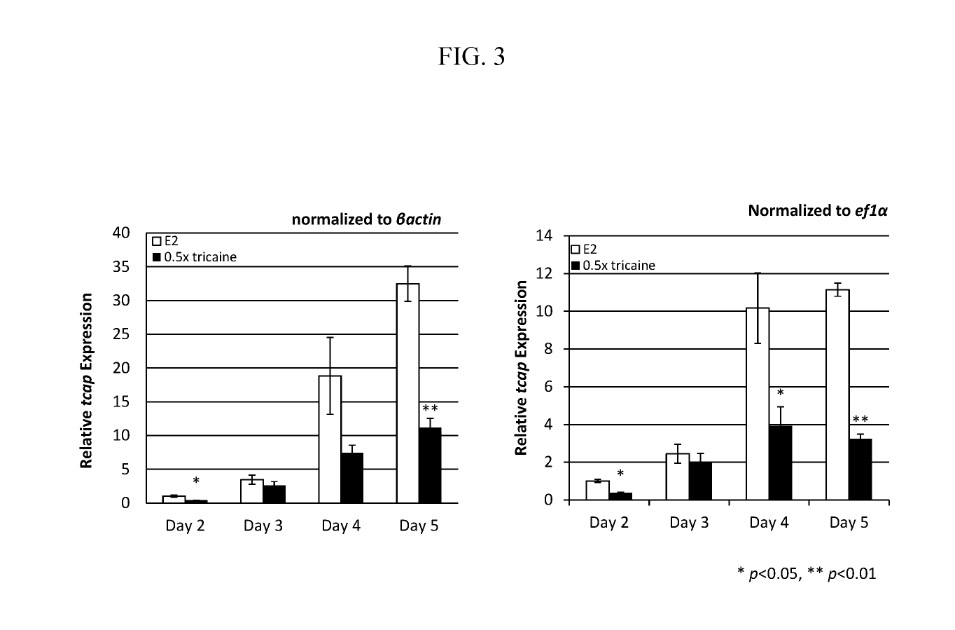

Wild type zebrafish at 2, 3, 4, and 5 dpf under normal condition (in E2 medium) and treatment with low dose tricaine, an anesthetic, were assessed for tcap expression using quantitative RT-PCR. The wild type zebrafish treated with tricaine exhibited significantly decreased Tcap expression as compared to the levels observed in wild type zebrafish under the normal conditions (FIGS. 3A and 3B). These results were obtained with blocked muscle activity indicating that suppressed locomotor activity with tricain treatment can increase tcap expression.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for increasing Tcap expression within a mammal, wherein said method comprises:
(a) identifying a mammal as being in need of increased Tcap expression, and
(b) administering a GAR1 inhibitor to said mammal under conditions wherein expression of Tcap within muscle cells of said mammal increases.

2. The method of claim 1, wherein said mammal is a human.

3. The method of claim 1, wherein said GAR1 inhibitor is an siRNA molecule.

4. A method for promoting muscle remodeling in a mammal, wherein said method comprises:
(a) identifying a mammal as being in need of muscle remodeling, and
(b) administering a GAR1 inhibitor to said mammal under conditions wherein muscle remodeling with said mammal is increased.

5. The method of claim 4, wherein said mammal is a human.

6. The method of claim 4, wherein said muscle remodeling is increased in the absence of muscle activation.

7. The method of claim 4, wherein said GAR1 inhibitor is an siRNA molecule.

8. A method for increasing Tcap expression within a mammal, wherein said method comprises:
(a) identifying a mammal as being in need of increased Tcap expression, and
(b) disrupting endogenous nucleic acid encoding a GAR1 polypeptide within said mammal under conditions wherein expression of biologically active GAR1 polypeptides is reduced and expression of Tcap within muscle cells of said mammal increases.

9. The method of claim 8, wherein said mammal is a human.

10. The method of claim 1, wherein said method comprises administering a TALEN or CRISPR/Cas9 nuclease system to said mammal to disrupt said endogenous nucleic acid.

11. A method for promoting muscle remodeling in a mammal, wherein said method comprises:
(a) identifying a mammal as being in need of muscle remodeling, and
(b) disrupting endogenous nucleic acid encoding a GAR1 polypeptide within said mammal under conditions wherein muscle remodeling with said mammal is increased.

12. The method of claim 11, wherein said mammal is a human.

13. The method of claim 11, wherein said method comprises administering a TALEN or CRISPR/Cas9 nuclease system to said mammal to disrupt said endogenous nucleic acid.

14. The method of claim 11, wherein said muscle remodeling is increased in the absence of muscle activation.
